(19) United States
(12) Patent Application Publication
(10) Pub. No.: US 2007/0167925 A1
(43) Pub. Date: Jul. 19, 2007

(54) Catheter with releasably coupled distal legs

(76) Inventor: William Jacqmein, Hopkinton, MA (US)

Correspondence Address: FAY KAPLUN & MARCIN, LLP, 15O BROADWAY, SUITE 702, NEW YORK, NY, 10038, US

(21) Appl. No.: 11/335,144

(22) Filed: Jan. 19, 2006

(51) Int. Cl. A61M 25/00

(52) U.S. Cl. 604/284

(57) ABSTRACT

A catheter comprising a proximal portion with first and second lumens extending therethrough and first and second legs extending from a distal end of the proximal portion, the first lumen extending through the first leg and the second lumen extending through the second leg in combination with a web of bioabsorbable material releasably coupling the first and second legs to one another.

### BACKGROUND OF THE INVENTION

Catheters are often used to withdraw and/or administer fluids (e.g., medications, blood products, nutrients, etc.). Multi-lumen, split tip catheters are often used for kidney dialysis in which blood is withdrawn from the vascular system, treated and returned to the vascular system. The two tips separate the openings for the withdrawal and return of fluids to improve flow rate, lower venous pressure, minimize the incidence of catheter occlusion and reduce recirculation of treated blood. In order to further separate the inflow and outflow openings of such a split tip catheter, the legs are generally of different length with, for example, an inflow opening being located proximally of an outflow opening to reduce recirculation.

However, movement of the tips often makes accurate placement difficult. Improper placement may cause problems including, for example, discomfort and reduced flow rate. Catheter placement often involves the use of a guidewire. A split tip catheter is often ‘woven’ over a previously placed guide wire via guide holes in the tips of the two catheter legs. After the guide wire is properly positioned, the catheter is ‘woven’ over the wire and slid over the wire to the desired position. The wire is then removed. However, the weaving process is time consuming and can cause issues on insertion as it increases an overall profile of the catheter. In addition, the shorter leg of the catheter may become caught in the vessel during insertion movement of the distal tips of the Y-shaped wire is makes accurate tracking and control of the guidewire more difficult. Thus, it is more difficult to place a split tip catheter and the accuracy of the placement of the catheter is also degraded.

### SUMMARY OF THE INVENTION

The present invention is directed to a catheter comprising a proximal portion with first and second lumens extending therethrough and first and second legs extending from a distal end of the proximal portion, the first lumen extending through the first leg and the second lumen extending through the second leg in combination with a web of bioabsorbable material releasably coupling the first and second legs to one another.

### BRIEF DESCRIPTION OF THE DRAWINGS

FIG. 1 is a top elevation view of a dual lumen split tip catheter that is encased in a biodegradable web, according to an embodiment of the present invention;

FIG. 2 is a front elevation view of the split tips encased in the biodegradable web;

FIG. 3 is a top elevation view showing the split tip catheter once the biodegradable web has dissolved.

### DETAILED DESCRIPTION

The present invention may be further understood with reference to the following description and the appended drawings, wherein like elements are referred to with the same reference numerals. Although the embodiments of the invention described relate to dialysis catheters, those skilled in the art will understand that the invention is equally applicable to a split tip catheter for any purpose.

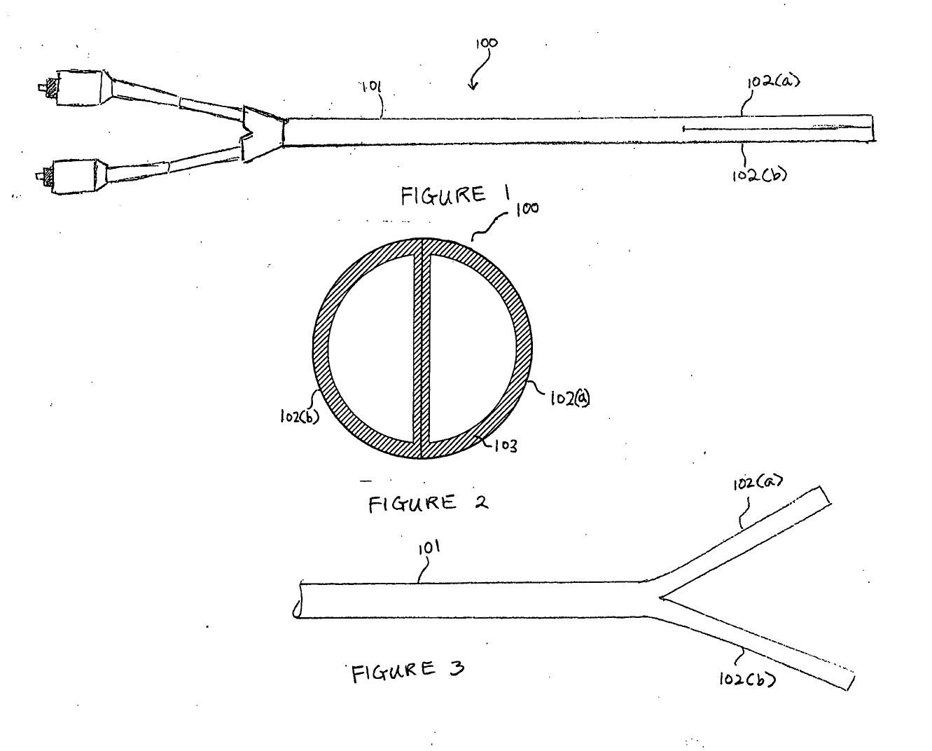

As shown in FIG. 1, a split tip catheter 100 according to an embodiment of the invention includes an elongate body 101 with two separate distal legs 102a and 102b releasably coupled to one another via a web 103 extending therebetween. The catheter 100 according to this embodiment, includes 2 D-shaped lumens 110 side by side separated from one another by a central wall 112. The central wall 112 is split in half along the length of the distal legs 102a and 102b with exterior surfaces 112′ of this split central wall 112 facing each other. The web 103 which couples these exterior surfaces 112′ to one another is formed of a biodegradable material such as polyethylene glycol (PEG), or other similarly bioabsorbable material so that a predetermined time after the web 103 contacts bodily fluids, the material dissolves, releasing the legs 102a and 102b from one another. Additional examples of suitable bioabsorbable materials include polysaccharaides (e.g., glucose, mannose, maltose, dextrose, fructose) water soluble salts (sodium chloride, sodium citrate, sodium phosphate) and polyvilnyl pirrolidone (polyacrylic acids). Those skilled in the art will understand that the time required for the web 103 to dissolve may be altered by changing the material of which the web 103 is formed, a configuration and/or size of the web 103. A size of the web 103 may be varied by selecting a length of legs 102a and 102b along which the web 103 extends. Having selected a length of the web 103, the configuration may be varied by selecting a number of segments into which the web 103 is divided. Depending on the application, anatomical considerations and physician preference, the desired dissolution time of the web 103 may vary from as short as one minute to 30 minutes or more.

The catheter 100 is inserted into the body over a previously placed guide wire (not shown) which is threaded through either of the legs 102a, 102b. Preferably the guide wire is threaded through whichever of the legs 102a, 102b is longest as this facilitates negotiating the anatomy of the vessel(s). Those skilled in the art will understand that any appropriately sized conventional guidewire may be used. When the catheter 100 has reached the desired position, the guide wire is removed. During insertion and while in the desired position, the web 103 contacts bodily fluids which, after a predetermined time dissolve the web 103 allowing the distal legs 102a and 102b to separate from one another.

FIG. 2 depicts a front elevation view of the distal legs 102a and 102b of the catheter 100 with the web 103 still intact. In this configuration, the distal legs 102a and 102b are connected to one another and unable to move independently. Although the catheter 100 is shown with a substantially circular cross-section, those skilled in the art will understand that the catheter 100 may be of any other cross-sectional shape such as elliptical, etc. FIG. 3 depicts the split tip catheter 100 with Web 103 completely dissolved and the distal legs 102a and 102b separated from one another.

The present invention has been described with reference to a specific embodiment, and more specifically to a dialysis catheter with dual lumen. However, other embodiments may be devised that are applicable to different medical devices, without departing from the scope of the invention. Accordingly, various modifications and changes may be made to the embodiments, without departing from the broadest spirit and scope of the present invention as set forth in the claims that follow. The specification and drawings are accordingly to be regarded in an illustrative rather than restrictive sense.

1. A catheter comprising:
a proximal portion with first and second lumens extending therethrough;
first and second legs extending from a distal end of the proximal portion, the first lumen extending through the first leg and the second lumen extending through the second leg; and
a web of bioabsorbable material releasably coupling the first and second legs to one another.

2. The catheter of claim 1, wherein the first and second legs, when coupled to one another, form a profile substantially similar to that of the proximal portion of the catheter.

3. The catheter of claim 1, wherein each of the first and second legs is substantially semi-circular in cross-section.

4. The catheter of claim 3, wherein each of the first and second legs includes a substantially planar wall and wherein the web extends between the substantially planar walls of the first and second legs binding them to one another.

5. The catheter of claim 4, wherein the web extends along substantially an entire length of the substantially planar walls of the first and second legs.

6. The catheter of claim 1, wherein the web includes a plurality of segments separated from one another.

7. The catheter of claim 1, wherein the web includes polyethylene glycol.

8. The catheter of claim 1, wherein the web is designed to dissolve contact a predetermined time after between the distal end of the catheter and bodily fluids.

9. The catheter of claim 4, wherein the web extends along only a portion of the length of the first and second legs.
